(19) United States
(12) Patent Application Publication
(10) Pub. No.: US 2025/0289618 A1
(43) Pub. Date: Sep. 18, 2025

(54) Labeling and Packaging System and Methods

(71) Applicant: C. R. Bard, Inc., Franklin Lakes, NJ (US)

(72) Inventors: Frederick Halden Simonsen, Marietta, GA (US); Tom Roberts, Bishop, GA (US)

(21) Appl. No.: 18/604,394

(22) Filed: Mar. 13, 2024

(51) Int. Cl. B65D 17/34; B65D 17/50

(52) U.S. Cl. CPC B65D 17/34; B65D 17/501; B65D 2203/02

(57) ABSTRACT

Labeling and packaging systems include a primary packaging with a top seam and a bottom seam, and enclosing a medical device within its interior cavity. The system further comprises a label system, which consists of a first body with a front surface and a rear surface, extending over a frontal plane and attached to the front surface of the primary packaging. Additionally, the label system includes a first right handle and a first left handle, both attached to the top edge of the first body. A tear line extends longitudinally from the top edge of the first body, and is disposed between the right handle and the left handle. The tear line has a distance (d) that is less than a length (L) of the first body. The tear line is designed to facilitate separation when the right handle is pulled in the opposite direction from the left handle.

### BACKGROUND

Labeling and packaging systems are commonly used in various industries to provide information and protection for products. In the field of medical devices, it is important to have efficient and secure packaging systems that ensure the integrity and safety of the devices during storage, transportation, and use. Previous approaches to labeling and packaging systems have focused on different aspects such as ease of opening, tamper-evident features, and integration with the primary packaging.

Previous approaches include applying labels directly onto the primary packaging, often using adhesives or similar means. These labels typically include printed information about the product, such as its name, instructions for use, and any relevant warnings or regulatory information. While these labels are widely used, they can sometimes be difficult to remove without damaging the primary packaging. Additionally, these labels can inhibit access to the contents of the packaging.

Other approaches include incorporating tear lines or perforations into the primary packaging itself. These tear lines are designed to facilitate opening of the packaging by providing a predetermined path along which the packaging can be torn. However, these tear lines may not always be user-friendly or intuitive, and there is a risk of accidental tearing or damage to the packaging during transport, storage, or the opening process causing the contents to spill onto the floor or a nonsterile surface, compromising the sterility of the medical device.

Disclosed herein are labeling and packaging systems and associated methods thereof directed to address the foregoing.

### SUMMARY

In some aspects, the techniques described herein relate to a labeling and packaging system including, a primary packaging having a top seam and a bottom seam and defining an interior cavity, a medical device disposed within the interior cavity, and a label system including, a first body extending over a frontal plane and defining a front surface and a rear surface and having a length (L) and a width (W), the rear surface coupled to a front surface of the primary packaging, a first right handle coupled to a right side of a top edge of the first body, a first left handle coupled to a left side of the top edge of the first body, and a tear line extending longitudinally from the top edge of the first body, between the first right handle and the first left handle, the tear line having a distance (d) that is less than the length (L) of the first body, the tear line configured to facilitate separation there along when the first right handle is urged in an opposite direction from the first left handle.

In some aspects, the techniques described herein relate to a system, wherein the distance (d) is between 20% and 80% of the length (L) of the first body.

In some aspects, the techniques described herein relate to a system, wherein the length (L) of the first body is equal to or less than a second length (L2) of the primary packaging.

In some aspects, the techniques described herein relate to a system, further including a tear stop disposed at a bottom end of the tear line

In some aspects, the techniques described herein relate to a system, wherein the tear stop includes a reinforcement displaying an increased tensile strength to prevent further separation of the first body.

In some aspects, the techniques described herein relate to a system, wherein the tear stop includes a frangible line angled relative to the tear line and configured to mitigate further separation along that tear line at a predetermined position on the primary packaging.

In some aspects, the techniques described herein relate to a system, further including a cut extending from the top edge of the first body and disposed between the first right handle and the first left handle, the cut extending through one or both of a portion of the first body and a portion of the top seam of the primary packaging.

In some aspects, the techniques described herein relate to a system, wherein the rear surface is coupled to the front surface of the primary packaging by one or more of adhesive sealing, mechanical sealing, energized sealing, heat sealing, and ultrasonic welding.

In some aspects, the techniques described herein relate to a system, further including a second right handle extending from a top edge of the first right handle and including a first fold line disposed therebetween, and a second left handle extending from a top edge of the first left handle and including a second fold line disposed therebetween, and a second body, coupled to both the second right handle and the second left handle along a first edge of the second body.

In some aspects, the techniques described herein relate to a system, wherein an outer perimeter of the second right handle aligns with an outer perimeter of the first right handle when folded along the first fold line, and an outer perimeter of the second left handle aligns with an outer perimeter of the first left handle when folded along the second fold line, and a surface of the second body is configured to couple with a rear side of the primary packaging.

In some aspects, the techniques described herein relate to a system, wherein the second body includes a second tear line extending from the first edge of the second body along a second distance (d2).

In some aspects, the techniques described herein relate to a method of opening a packaging and label system including, providing a primary packaging having a top seam and a bottom seam and defining an interior cavity, a medical device disposed within the interior cavity, and a label system having a body, a rear surface of the body coupled with a front surface of the primary packaging, grasping a first right handle and a first left handle of the label system, the first right handle coupled to a right side of a top edge of the body, and the first left handle coupled to a left side of the top edge of the body, urging the first right handle in an opposite direction from the first left handle along an axis extending at an angle relative to a longitudinal axis of the primary packaging, separating the body along a tear line extending longitudinally, and tearing a portion of the primary packaging to access the medical device.

In some aspects, the techniques described herein relate to a method, wherein in the tear line is a line of weakness extending along the body by a distance (d) to facilitate separation therealong, the distance (d) being less than a longitudinal length (L) of the body.

In some aspects, the techniques described herein relate to a method, wherein the distance (d) of the tear line is less than a longitudinal length (L2) of the primary packaging.

In some aspects, the techniques described herein relate to a method, further including a tear stop disposed at a bottom end of the tear line and configured to prevent further separation of the body beyond the distance (d).

In some aspects, the techniques described herein relate to a system, wherein the tear stop includes one of a reinforcement or a frangible line angled relative to the tear line and configured to mitigate further separation along that tear line at a predetermined position on the primary packaging.

In some aspects, the techniques described herein relate to a method, wherein coupling the rear surface of the body to the front surface of the primary packaging includes one or more of adhesive sealing, mechanical sealing, energized sealing, heat sealing, and ultrasonic welding.

In some aspects, the techniques described herein relate to a method of manufacturing a label and packaging system including, forming a primary packaging having a top seam and a bottom seam, a front surface and a rear surface, the primary packaging defining an interior cavity containing a medical device, and coupling a rear surface of a body of a labeling system to the front surface of the primary packaging, the labeling system including a first right handle extending from a right side of a top edge of the body, and a first left handle extending from a left side of the top edge of the body.

In some aspects, the techniques described herein relate to a method, further including folding a second right handle relative to the first right handle along a first fold line such that an outer perimeter of the second right handle aligns with the outer perimeter of the first right handle, and folding a second left handle relative to the first left handle along a second fold line such that an outer perimeter of the second left handle aligns with the outer perimeter of the first left handle.

In some aspects, the techniques described herein relate to a method, further including coupling a surface of a second body to the rear surface of the primary packaging.

In some aspects, the techniques described herein relate to a method, wherein coupling the rear surface of the body to the front surface of the primary packaging includes one or more of adhesive sealing, mechanical sealing, energized sealing, heat sealing, and ultrasonic welding.

In some aspects, the techniques described herein relate to a method, further including forming a cut disposed between the first right handle and the first left handle and extending from the top edge of the body, the cut extending through one or both of the body and a portion of the top seam.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exploded view of a label and packaging system generally including a label system, a primary packaging, and a medical device, in accordance with embodiments disclosed herein.

FIGS. 2A-2B show perspective views of the label and packaging system of FIG. 1 in an assembled state, in accordance with embodiments disclosed herein.

FIG. 2C shows a perspective view of a label system without handles and a packaging system in an assembled state, in accordance with embodiments disclosed herein.

FIG. 2D shows a perspective view of a label system without handles and with a frangible tear stop in an assembled state with a packaging system, in accordance with embodiments disclosed herein.

FIG. 2E shows a perspective view of a label system with an offset tear line in an assembled state with a packaging system, in accordance with embodiments disclosed herein.

FIGS. 3A-3B show foldable label systems in an unfolded configuration, in accordance with embodiments disclosed herein.

FIG. 3C shows a foldable label system in a folded configuration, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “right,” “left,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

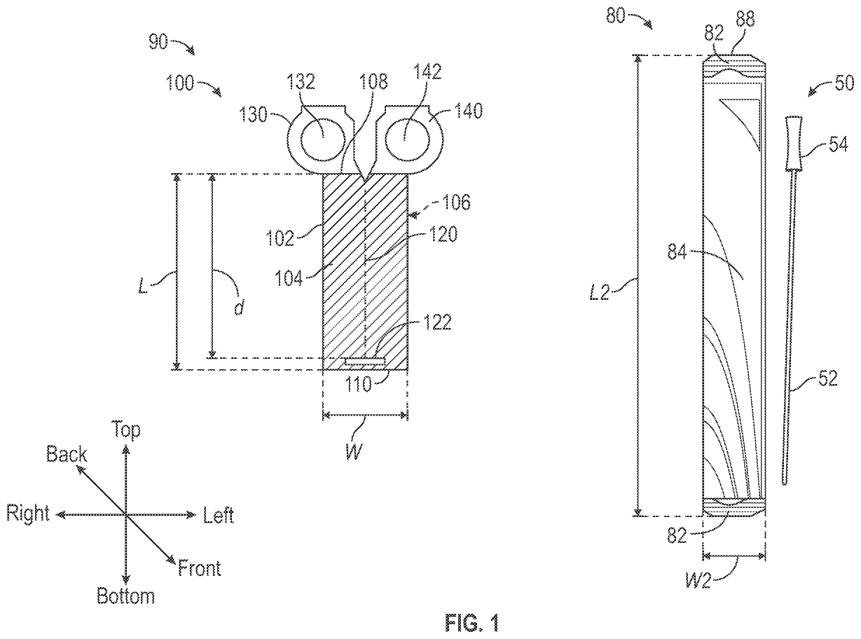

To assist in the description of embodiments described herein, as shown in FIG. 1, a sagittal axis extends between the front and the back. A longitudinal axis extends between the top and the bottom. A frontal axis extends between the right and the left. A frontal plane is defined by the longitudinal axis and the frontal axis. A sagittal plane is defined by the longitudinal axis and the sagittal axis. A transverse plane is defined by the frontal axis and the sagittal axis.

Unless defined otherwise, all technical and scientific terms used herein have the same meaning as commonly understood by those of ordinary skill in the art.

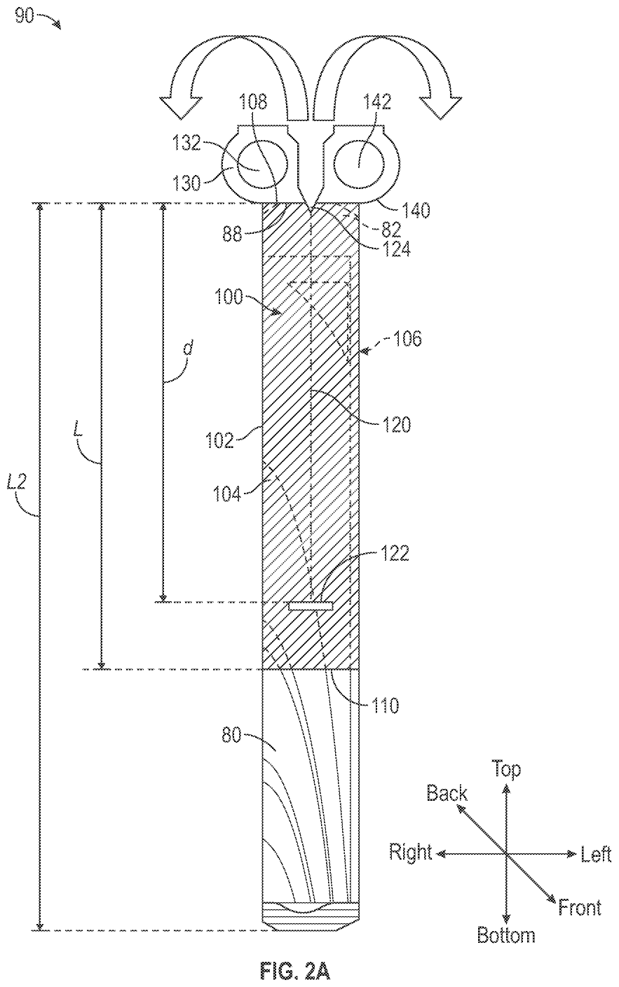

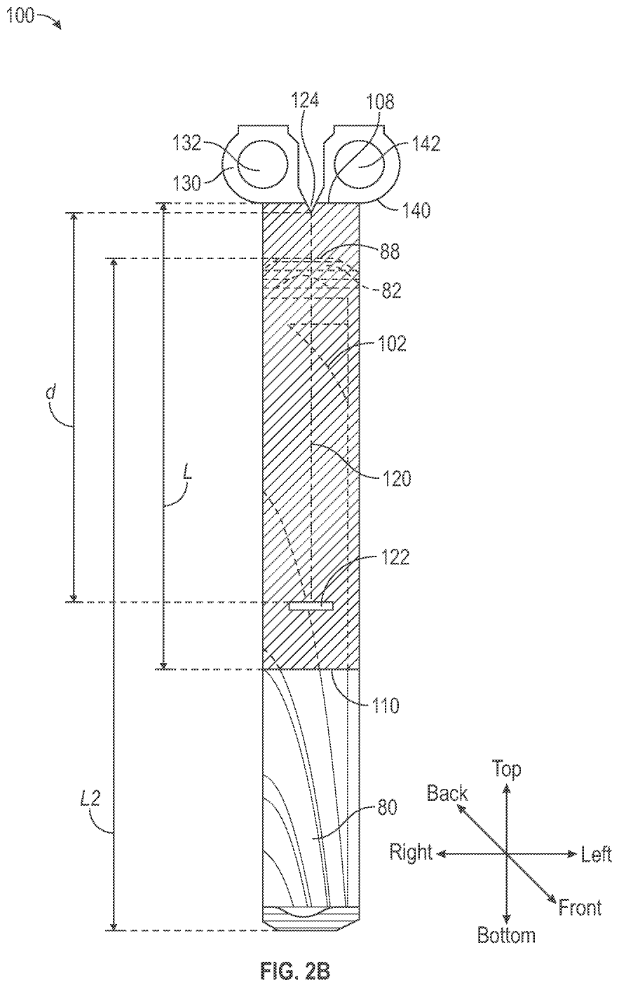

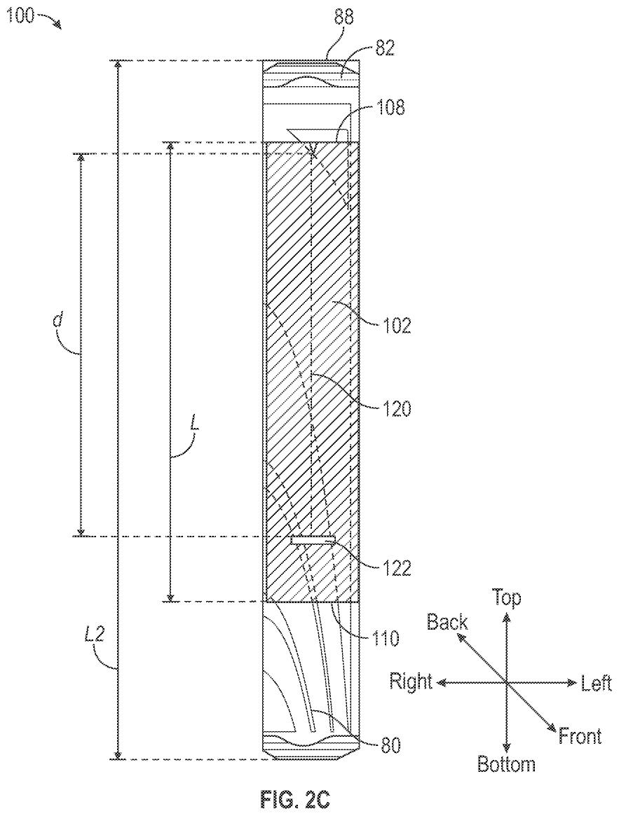

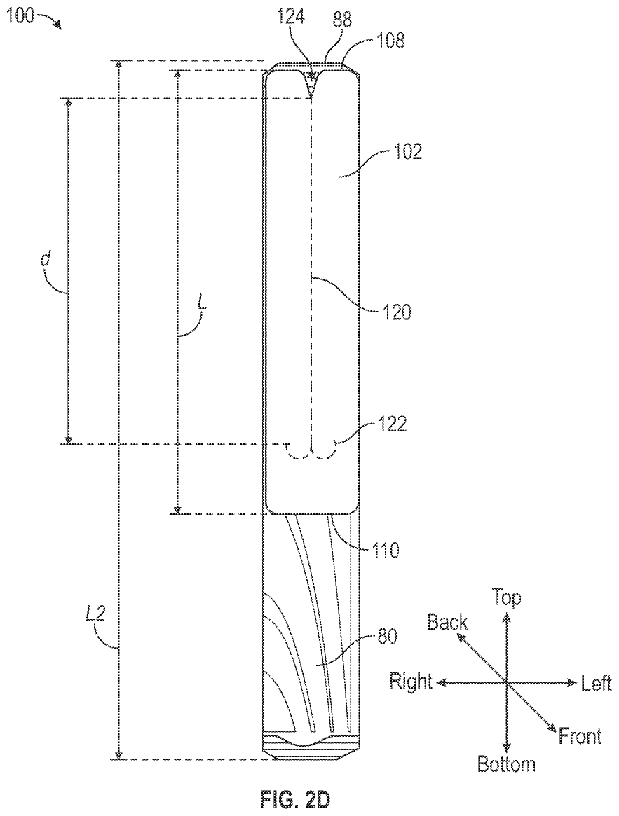

FIG. 1 shows an exploded view of a label and packaging system 90 generally including a label system 100, a primary packaging 80 and a medical device 50. Exemplary medical devices can include catheters, intermittent catheters, Foley catheters, balloon catheters, indwelling catheters, peritoneal catheters, pleural drainage catheters, or the like. However, these examples are not intended to be limiting and it will be appreciated that other products or types of medical devices are also contemplated. The primary packaging 80 is configured to contain the medical device 50 therein and the label system 100 is configured to couple with the primary packaging. FIG. 2A shows the label and packaging system 90 in an assembled state.

In an embodiment, the primary packaging 80 is formed of one or more layers of material folded and sealed along one or more seams 82 to define an interior cavity therein. In an embodiment, the interior cavity can define a sterile environment. The one or more layers of material can include the same material or different materials. Exemplary materials of the one or more layers of material include organic or synthetic materials, woven or non-woven materials, plastic, polymers, metal, alloys, composites, foils, paper, wax paper, combinations thereof, or the like.

In an embodiment, the one or more layers of material define a fluid impermeable, liquid impermeable, and/or gaseous impermeable layer. For example, the one or more layers of material define a water impermeable layer and/or an oxygen impermeable layer. As such, the medical device 50 can be maintained in a sterile and/or wetted environment during storage and transport up until the primary package 80 is opened. In an embodiment, the one or more seams 82 can be secured using adhesive sealing, mechanical sealing, energized sealing such as heat sealing, ultrasonic welding, combinations thereof, or the like.

In an embodiment, the medical device 50 includes a sterile portion 52 configured to be disposed within the body of the patient, and a non-sterile portion 54 configured to be handled by the user to facilitate manipulating the medical device 50 without touching the sterile portion 52. For example, where the medical device 50 is an intermittent catheter, the sterile portion 52 can be the catheter tube, configured to be disposed within the urethra or bladder of the patient. The non-sterile portion 54 can be a finger grip or funnel configured to be grasped by the user to facilitate manipulating the medical device 50 without having to touch the catheter tube, sterile portion 52. In an embodiment, the medical device 50 is oriented within the primary packaging 80 with the non-sterile portion 54 positioned adjacent a top edge 88 of the primary packaging. As described in more detail herein, opening the primary packaging 80 can involve tearing a portion of the primary packaging 80. In an embodiment, tearing the primary packaging can begin proximate the top edge 88 to provide access to the non-sterile portion 54 first while maintaining the sterile portion 52 within the primary packaging 80.

In an embodiment, the label system 100 generally includes a body 102 extending over a frontal plane and defining a front surface 104 and a back surface 106. The body 102 defines a length (L) and a width (W). The label system 100 further includes a tear line 120 extending along a longitudinal axis from a top edge 108 towards a bottom edge 110, to a point that is above the bottom edge 110 of the body 102. In an embodiment, the tear line 120 extends along a distance (d) that is less than the length (L) of the body 102. In an embodiment, the tear line 120 extends a distance (d) that is between 20% and 80% of the length (L) of the body 102. However, it will be appreciated that greater or lesser distances are also contemplated.

In an embodiment, as shown in FIG. 2A, the label system 100 can be coupled with the primary packaging 80 with a central longitudinal axis of the body 102 aligning with a central longitudinal axis of the primary packaging 80. In an embodiment, the label system 100 can be coupled with the primary packaging 80 in an offset position, with a central longitudinal axis of the body 102 disposed to either the right side or the left side of the central longitudinal axis of the primary packaging 80. In an embodiment, the left edge of the body 102 can be aligned with the left edge of the primary packaging 80. In an embodiment, the right edge of the body 102 can be aligned with the right edge of the primary packaging 80.

The tear line 120 is a die cut line, laser cut line, perforation, frangible line, score line, groove, or similar line of weakness configured to facilitate separation of the body 102 therealong, as described in more detail herein. In an embodiment, the tear line 120 is separated along an entire length (distance (d)) of the tear line 120. In an embodiment, the tear line 120 is separated along a portion or a plurality of portions along the distance (d) of the tear line 120, e.g. to form a frangible perforation, or the like. In an embodiment, the tear line 120 is intact along the distance (d) of the tear line 120, e.g. to form a score line, groove line, or the like. As shown, the tear line 120 extends linearly, however, other shaped tear lines are also contemplated including curved, wavy, closed curve, or the like. In an embodiment, as shown in FIG. 2A, the tear line 120 extends parallel to the longitudinal axis of the body 102. In an embodiment, the tear line 120 extends at an angle to the longitudinal axis of the body 102.

In an embodiment, as shown in FIG. 2A the label system 100 includes a reinforcement 122 tear stop 122 disposed between a bottom of the tear line 120 and the bottom edge 110 of the body 102. The tear stop 122 is configured to prevent further separation of the body 102 along the tear line 120. In an embodiment, the tear stop 122 is a reinforced portion made of the same material as the body 102. In an embodiment, the tear stop 122 is a reinforced portion made of a different material from the body 102 having an increased tensile strength to mitigate or prevent tearing.

In an embodiment, as shown in FIG. 2D, the tear stop 122 includes one or more frangible lines such as a die cut line, laser cut line, perforation, frangible line, score line, groove, or similar line of weakness as described herein extending at an angle relative to the axial length of the tear line 120. For example, as shown in FIG. 2D, the tear stop 122 includes a first frangible line extending from the bottom end of the tear line 120 through a first arc, and a second frangible line extending from the bottom end of the tear line 120 through a second arc in an opposite direction from the first arc. One or both of the first arc and the second arc can extend through an arc radius of between 5° degrees and 275°. However it will be appreciated that greater or lesser arc radii are also contemplated. It will also be appreciated that other numbers, angles, shapes, and configurations of frangible lines are also contemplated to be included in the tear stop 122. Advantageously, the frangible line tear stop 122 redirects the direction of the tear, from the axis of the tear line 120 to prevent further separation of the packaging 80 therealong. For example, as shown in FIG. 2D, the semi-circular shape of the tear stop 122 redirects the direction of the tear back on itself to mitigate further tearing momentum along the axis of the tear line 120 and arrest the separation of the primary packaging 80 at a predetermined location thereon.

The label system 100 further includes a right handle 130 and a left handle 140 each extending upwards from the top edge 108 of the body 102. The right handle 130 is coupled with a right side of the top edge 108 of the body 102 and the left handle 140 is coupled with a left side of the top edge 108 of the body 102. In an embodiment one or both of the right handle and the left handle are formed integrally with the body 102 as a single monolithic piece. In an embodiment one or both of the right handle and the left handle are formed separately and coupled with the body 102 using adhesive sealing, mechanical sealing, energized sealing such as heat sealing, ultrasonic welding, combinations thereof, or the like. In an embodiment one or more of the right handle, left handle and the body 102 are formed of the same material. In an embodiment one or more of the right handle, left handle and the body 102 are formed of different materials.

Each of the right handle 130 and the left handle 140 are configured to facilitate grasping the body 102 by a user. For example, as shown in FIG. 1, the right handle 130 defines a right finger loop having an aperture 132 and configured to receive a first digit of a user therethrough. Similarly the left handle 140 defines a left finger loop having an aperture 142 and configured to receive a second digit of a user therethrough. It will be appreciated, however, the finger loop handles of the right handle 130 and the left handle 140 are not intended to be limiting and that other shaped handles for the right handle 130 and the left handle 140 are also contemplated. Exemplary handles include a handle having two or more apertures each configured to receive at least a digit of the user, a finger loop configured to receive two or more digits, straight handles, curved handles, T-shaped handles, combinations thereof, or the like.

As shown in FIGS. 2A-2C, in an embodiment, the body 102 can be coupled to the primary packaging 80. For example, the back surface 106 of the body 102 can be coupled with a front surface 84 of the primary packaging 80 using adhesive sealing, mechanical sealing, energized sealing such as heat sealing, ultrasonic welding, combinations thereof, or the like. In an embodiment, as shown in FIG. 2A, the top edge 108 of the body 102 can be aligned with the top edge 88 of the primary packaging 80. In an embodiment, as shown in FIG. 2B, the top edge 108 of the body 102 can be positioned above the top edge 88 of the primary packaging 80, i.e., the body 102 can be positioned such that the top edge 108 extends beyond the top edge 88 of the primary packaging 80. In an embodiment, as shown in FIG. 2C, the top edge 108 of the body 102 can be positioned below the top edge 88 of the primary packaging 80, i.e., the body 102 can be positioned such that the top edge 88 of the primary packaging 80 extends beyond the top edge 108 of the body 102. In an embodiment, as shown in FIG. 2C the label system 100 can be provided without one or both of the right handle 130 and the left handle 140 and include one or more of the body 102, the tear line 120, and the tear stop 122.

In an embodiment, a length (L) of the body 102 is equal to or less than a length (L2) of the primary packaging 80. For example, the length (L) of the body 102 can be between 10% and 100% of the length (L2) of the primary packaging 80. However, it will be appreciated that greater or lesser lengths (L) of the body 102 are also contemplated. In an embodiment, a width (W) of the body 102 is equal to or less than a width (W2) of the primary packaging 80. For example, a width (W) of the body 102 can be between 10% and 100% of the width (W2) of the primary packaging 80. However, it will be appreciated that greater or lesser width (W) of the body 102 are also contemplated.

In an exemplary method of manufacture, a primary packaging 80 is provided defining an interior cavity that defines a sterile environment and includes a medical device 50 disposed therein. A label system 100 is coupled to the primary packaging by coupling a surface of the body 102 of the label system 100 with a surface of the primary packaging 80. A top edge 108 of the body is aligned with a top edge 88 of the primary packaging 80.

In an embodiment, a cut 124 can be made through one or both of a portion of the seal 82 of the primary packaging 80 and a portion of the body 102. The cut 124 extends downwards from the top edge 88 of the primary packaging 80, and/or the top edge 108 of the body 102 and is aligned with the tear line 120 and/or disposed between the right handle 130 and the left handle 140. The cut 124 extends to a point that is above the bottom edge of the seal 82. In an embodiment, the cut 124 extends a distance that is less than half the height of the seal 82. As such the cut 124 does not compromise the sterile environment defined by the primary packaging 80 but can facilitate opening the primary packaging as described in more detail herein. In an embodiment, the cut line 124 is disposed 1.5 cm from one of the left edge or the right edge of the body 102 or the primary packaging 80. However, it will be appreciated that greater or lesser distances are also contemplated. In an embodiment, the cut line 124 extends 0.5 cm from a top edge 88. However, it will be appreciated that greater or lesser distances are also contemplated. In an embodiment, the cut 124 defines a straight line cut, or a shaped notch such as a triangular notch or “V”-notch, a square notch, a rectangular notch, a semi-circular notch, or similar suitably shaped notch without limitation.

In an embodiment, as shown in FIGS. 1 and 2A, one or both of the tear line 120 and a cut line 124 are disposed centrally on the body 102, equidistant from a right edge and a left edge of the body 102. In an embodiment, one or both of the tear line 120 and the cut line 124 can be disposed to either a left side or a right side of the central position on the body 102, i.e., disposed closer to a left edge or closer to a right edge of the body 102. For example, as shown in FIG. 2E, the tear line 120 and the cut line 124 are disposed to a left side of a central longitudinal axis 70 of the label system 100.

In an exemplary method of use, as shown in FIG. 2A, a label and packaging system 90 is provided including a label system 100 coupled to a primary packaging 80, as described herein. A medical device 50 is contained within the primary packaging 80 in a sterile environment. A user can grasp the label system 100 by grasping the right handle 130 and the left handle 140. For example, a user can place a first digit through the aperture 132 of the right handle 130 and a second digit through the aperture 142 of the left handle 140. The user can then urge the right handle 130 and the left handle 140 in opposite directions, for example in opposite directions along the frontal axis, the sagittal axis, or along an axis extending at an angle thereto. One or both of the cut 124 and the tear line 120 facilitate separation of a right portion of the body 102 and a left portion of the body 102 as the right handle 130 and the left handle 140 are urged in opposite directions. One or both of the cut 124 and the tear line 120 facilitate separation of a right portion of the primary packaging 80 and a left portion of the packaging 80 as the right handle 130 and the left handle 140 are urged in opposite directions.

A user can continue to separate the body 102 and the packaging 80 along the tear line 120 up until the bottom end of the tear line 120 is reached. In an embodiment, the body 102 further includes a tear stop 122 to prevent further separation beyond the bottom end of the tear line 120. Advantageously, the tear line 120 and optionally the tear stop 122 guides the separation of the body 102 and the primary packaging 80 to a predetermined distance, e.g., distance (d). This allows the user to open the primary packaging 80 and access the medical device 50 while still allowing the primary packaging 80 to retain the medical device 50 therein. For example, if the packaging 80 were allowed to separate along a majority or an entire length (L2) of the packaging 80, the medical device 50 would fall out of the packaging 80 and compromise the sterility of the medical device 50, e.g., by falling on the floor or a non-sterile surface. Further, the user would be unable to return the medical device 50 to the primary packaging 80 after use to contain any mess and facilitate disposal. By contrast, the label system 100 restricts separation to a predefined length and/or path, maintaining a portion of the primary packaging 80 intact to define a pocket and to retain the medical device 50 therein.

In an embodiment, the body 102 includes an adhesive disposed on a surface of the body 102, e.g., the front surface 104, and configured to adhere the label system 100 and primary packaging 80 assembly to a surface for ease of use. For example, once the primary packaging 80 has been opened, as described herein, the adhesive front surface 104 allows the label and packaging system 90 to be adhered to a wall at a convenient position to facilitate usage of the medical device 50. In an embodiment, the adhesive front surface 104 facilitates attaching the label and packaging system 90 to a surface to prevent the label system 100 and primary packaging 80 assembly from falling off the surface. In an embodiment, the adhesive surface disposed on the front surface 104 further includes a release layer (not shown) disposed thereon and configured to protect the adhesive surface during transport and storage.

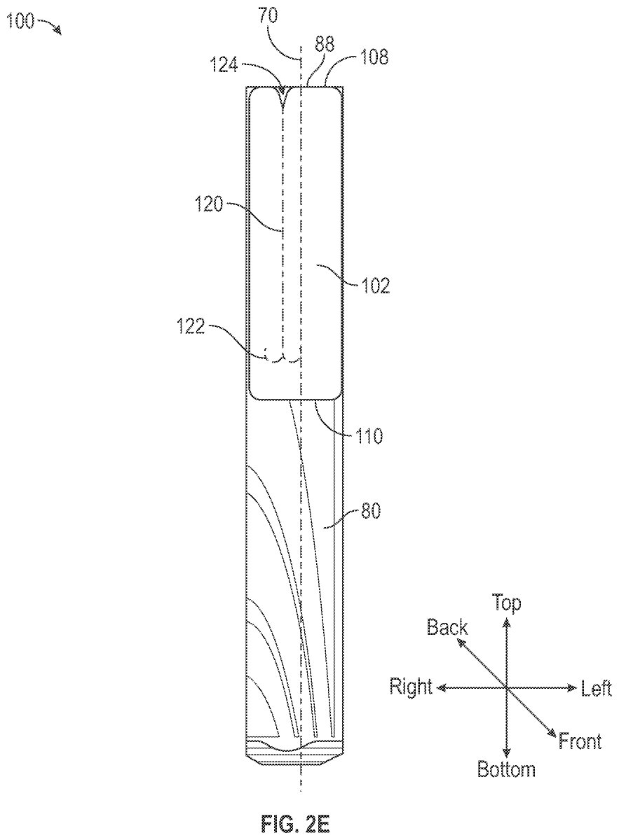

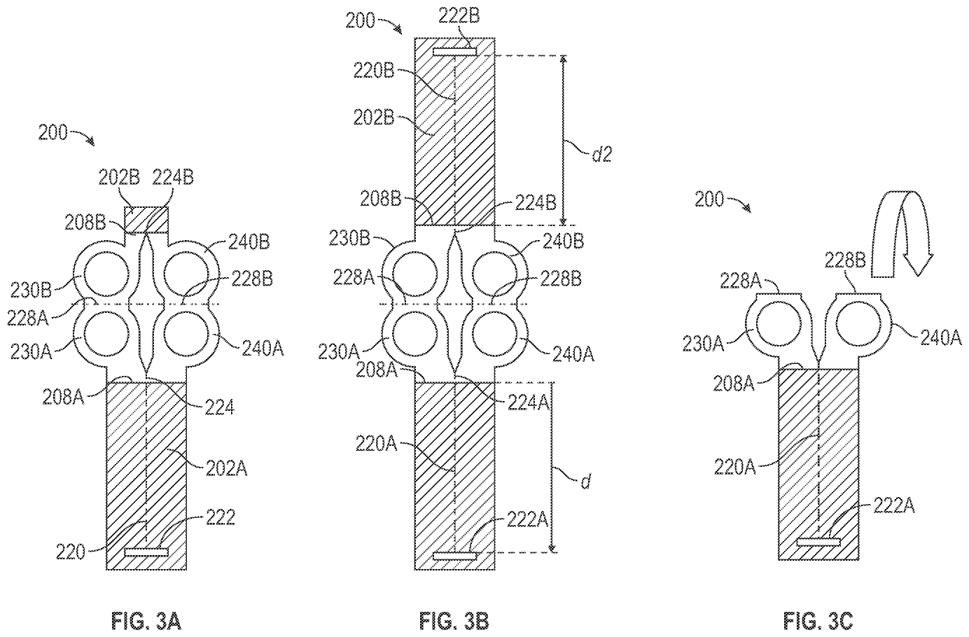

FIGS. 3A-3C show an embodiment of a label system 200 for use with a primary packaging 80 and medical device 50, as described herein. The label system 200 includes a first body 202A, a first right handle 230A, and a first left handle 240A. The first body 202A includes a first tear line 220A extending from a top edge 208A of the first body 202A. The label system 200 further includes a second right handle 230B extending from a top edge of the first right handle 230A and a second left handle 240B extending from a top edge of the first left handle 240A. The label system 200 includes a first fold line 228A extending along a frontal axis and disposed equidistant between the first right handle 230A and the second right handle 230B, and a second fold line 228B disposed equidistant between the first left handle 240A and the second left handle 240B and extending along a frontal axis. The second right handle 230B and the second left handle 240B are coupled to a first edge 208B of the second body 202B.

In an embodiment, the label system 200 can be folded along one or both of the first fold line 228A and the second fold line 228B so that the first right handle 230A aligns with the second right handle 230B, the first left handle 240A aligns with the second left handle 240B and the second body aligns 202B aligns with the first body 202A. For example, one or more of an outer perimeter of the first right handle 230A aligns with the outer perimeter of the second right handle 230B, an outer perimeter of the first left handle 240A aligns with the outer perimeter of the second left handle 240B, an aperture of the first right handle 230A aligns with an aperture of the second right handle 230B, an aperture of the first left handle 240A aligns with the aperture of the second left handle 240B, and the first edge 208B of the second body 202B aligns with the top edge 208A of the first body 202A, or combinations thereof.

In an embodiment, a surface of the first right handle 230A is coupled with a surface of the second right handle 230B, and a surface of the first left handle 240A is coupled with a surface of the second left handle 240B, using adhesive sealing, mechanical sealing, energized sealing such as heat sealing, ultrasonic welding, combinations thereof, or the like. In an embodiment, a surface of the first body 202A is coupled with a front surface of the primary packaging 80 and a surface of the second body 202B is coupled with a rear surface of the primary packaging 80 using adhesive sealing, mechanical sealing, energized sealing such as heat sealing, ultrasonic welding, combinations thereof, or the like. For example, one or both of the first body 202A and the second body 202B includes an adhesive disposed on a surface thereof. As shown in FIG. 3C, as the label system 200 is folded along the first fold line 228A and the second fold line 228B, the first body 202A can be adhered to a front side of the primary packaging 80 and the second body 202B can be adhered to the rear side of the primary packaging 80.

In an embodiment, as shown in FIG. 3A, the first body 202A and the second body 202B can be of different dimensions, i.e., a length and/or width that are different from the length (L) and/or width (W) of the first body 202A. In an embodiment, as shown in FIG. 3B, the first body 202A and the second body 202B can be of the same dimensions, i.e., having a length (L) and/or width (W) that are the same. In an embodiment, as shown in FIG. 3A, the first body 202A can include one or more of a tear line 220, a tear stop 222 and a cut 224.

In an embodiment, as shown in FIG. 3B, the first body 202A includes one or more of a first tear line 220A, a first tear stop 222A, and a first cut 224A and the second body 202B includes one or more of a second tear line 220B, a second tear stop 222B, and a second cut 224B. In an embodiment, the second tear line 220B extends along a second distance (d2). The second distance of the second tear line 220B can be the same or different from the distance (d) of the first tear line 220A.

In an exemplary method of use, as shown in FIG. 3C, the first right handle 230A and the second right handle 230B can be moved in an opposite direction from the first left handle 240A and the second left handle 240B to tear both the first body 202A and the second body 202B along at least the first tear line 220A to separate both the first body 202A and the second body 202B and the primary packaging 80 disposed therebetween.

Advantageously, the label system(s) 100, 200 can be applied to the primary packaging 80 after formation of the primary packaging 80. This allows the labeling system, which facilitates opening of the primary packaging, to be added at a later time, e.g., retrofitted to existing products before sale, or fitted to the packaging by an end user that has reduced dexterity. The label system(s) can be modified to display different information about the primary packaging 80 and/or medical device 50 disposed therein, and fitted to the primary packaging 80 after formation of the primary packaging 80. Exemplary information can include information about the brand, make, model, serial number, batch number of the medical device, instructions for use, instructions on how to open, instructions for disposal, or the like. Advantageously, the label system(s) 100, 200 can include instructions printed thereon to indicate how to use the label system(s) 100, 200 to open the primary packaging 80, e.g. to assist in which end to open the primary packaging 80, where to start the separation along the tear line 120, or at cut line 124, how to hole finger handle 130, 140, etc.

Advantageously, a single type of label system, e.g., one of the label systems 100, 200, can be coupled to various types, sizes, and shapes, of primary packaging 80 simplifying manufacturing and reducing associated costs. Advantageously, the labeling system(s) 100, 200 can indicate to a user which is the correct end to open the primary packaging 80, i.e., to begin opening the primary packaging 80 from a top edge 88, adjacent the non-sterile portion 54 of the medical device 50. This mitigates a user accidentally opening the primary packaging 80 from a different edge that is not adjacent the non-sterile portion 54 and compromising the sterility of the sterile portion 52.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A labeling and packaging system, comprising:
a primary packaging having a top seam and a bottom seam and defining an interior cavity;
a medical device disposed within the interior cavity; and
a label system, comprising:
a first body extending over a frontal plane and defining a front surface and a rear surface and having a length (L) and a width (W), the rear surface coupled to a front surface of the primary packaging;
a first right handle coupled to a right side of a top edge of the first body;
a first left handle coupled to a left side of the top edge of the first body; and
a tear line extending longitudinally from the top edge of the first body, between the first right handle and the first left handle, the tear line having a distance (d) that is less than the length (L) of the first body, the tear line configured to facilitate separation there along when the first right handle is urged in an opposite direction from the first left handle.

2. The system according to claim 1, wherein the distance (d) is between 20% and 80% of the length (L) of the first body.

3. The system according to claim 2, wherein the length (L) of the first body is equal to or less than a second length (L2) of the primary packaging.

4. The system according to claim 1, further including a tear stop disposed at a bottom end of the tear line

5. The system according to claim 4, wherein the tear stop includes a reinforcement displaying an increased tensile strength to prevent further separation of the first body.

6. The system according to claim 4, wherein the tear stop includes a frangible line angled relative to the tear line and configured to mitigate further separation along that tear line at a predetermined position on the primary packaging.

7. The system according to claim 1, further including a cut extending from the top edge of the first body and disposed between the first right handle and the first left handle, the cut extending through one or both of a portion of the first body and a portion of the top seam of the primary packaging.

8. The system according to claim 1, wherein the rear surface is coupled to the front surface of the primary packaging by one or more of adhesive sealing, mechanical sealing, energized sealing, heat sealing, and ultrasonic welding.

9. The system according to claim 1, further including a second right handle extending from a top edge of the first right handle and including a first fold line disposed therebetween, and a second left handle extending from a top edge of the first left handle and including a second fold line disposed therebetween, and a second body, coupled to both the second right handle and the second left handle along a first edge of the second body.

10. The system according to claim 9, wherein an outer perimeter of the second right handle aligns with an outer perimeter of the first right handle when folded along the first fold line, and an outer perimeter of the second left handle aligns with an outer perimeter of the first left handle when folded along the second fold line, and a surface of the second body is configured to couple with a rear side of the primary packaging.

11. The system according to claim 9, wherein the second body includes a second tear line extending from the first edge of the second body along a second distance (d2).

12. A method of opening a packaging and label system, comprising:
providing a primary packaging having a top seam and a bottom seam and defining an interior cavity, a medical device disposed within the interior cavity, and a label system having a body, a rear surface of the body coupled with a front surface of the primary packaging;
grasping a first right handle and a first left handle of the label system, the first right handle coupled to a right side of a top edge of the body, and the first left handle coupled to a left side of the top edge of the body;
urging the first right handle in an opposite direction from the first left handle along an axis extending at an angle relative to a longitudinal axis of the primary packaging;
separating the body along a tear line extending longitudinally; and
tearing a portion of the primary packaging to access the medical device.

13. The method according to claim 12, wherein in the tear line is a line of weakness extending along the body by a distance (d) to facilitate separation therealong, the distance (d) being less than a longitudinal length (L) of the body.

14. The method according to claim 13, wherein the distance (d) of the tear line is less than a longitudinal length (L2) of the primary packaging.

15. The method according to claim 13, further including a tear stop disposed at a bottom end of the tear line and configured to prevent further separation of the body beyond the distance (d).

16. The method according to claim 15, wherein the tear stop includes one of a reinforcement or a frangible line angled relative to the tear line and configured to mitigate further separation along that tear line at a predetermined position on the primary packaging.

17. The method according to claim 12, wherein coupling the rear surface of the body to the front surface of the primary packaging includes one or more of adhesive sealing, mechanical sealing, energized sealing, heat sealing, and ultrasonic welding.

18. A method of manufacturing a label and packaging system, comprising:
forming a primary packaging having a top seam and a bottom seam, a front surface and a rear surface, the primary packaging defining an interior cavity containing a medical device; and
coupling a rear surface of a body of a labeling system to the front surface of the primary packaging, the labeling system including a first right handle extending from a right side of a top edge of the body, and a first left handle extending from a left side of the top edge of the body.

19. The method according to claim 18, further including folding a second right handle relative to the first right handle along a first fold line such that an outer perimeter of the second right handle aligns with the outer perimeter of the first right handle, and folding a second left handle relative to the first left handle along a second fold line such that an outer perimeter of the second left handle aligns with the outer perimeter of the first left handle.

20. The method according to claim 19, further including coupling a surface of a second body to the rear surface of the primary packaging.

21. The method according to claim 19, wherein coupling the rear surface of the body to the front surface of the primary packaging includes one or more of adhesive sealing, mechanical sealing, energized sealing, heat sealing, and ultrasonic welding.

22. The method according to claim 18, further including forming a cut disposed between the first right handle and the first left handle and extending from the top edge of the body, the cut extending through one or both of the body and a portion of the top seam.
